(19) United States
(12) Patent Application Publication
(10) Pub. No.: US 2025/0345400 A1
(43) Pub. Date: Nov. 13, 2025

(54) METHODS FOR TREATMENT OF COGNITIVE IMPAIRMENT IN CIRRHOSIS PATIENTS

(71) Applicant: VIRGINIA COMMONWEALTH UNIVERSITY, Richmond, VA (US)

(72) Inventor: Jasmohan BAJAJ, RICHMOND, VA (US)

(21) Appl. No.: 18/864,593

(22) PCT Filed: May 17, 2023
(86) PCT No.: PCT/US2023/022518

§ 371 Date: Nov. 11, 2024

Related U.S. Application Data
(60) Provisional application No. 63/343,144, filed on May 18, 2022.

(51) Int. Cl. A61K 38/38; A61P 25/00

(52) U.S. Cl. CPC A61K 38/38; A61P 25/00

(57) ABSTRACT

Provided are methods of treating cognitive impairment in a subject suffering from cirrhosis, comprising administering to the subject a therapeutically effective amount of albumin, wherein the subject does not currently suffer from overt hepatic encephalopathy. In particular, the subject has not suffered from overt hepatic encephalopathy within one month of the administering step. The albumin may be administered intravenously.

### FIELD OF THE INVENTION

The disclosure provides methods for treating cognitive impairment that persists despite treatment in cirrhosis patients without overt hepatic encephalopathy by administering albumin.

### BACKGROUND

Overt hepatic encephalopathy (OHE) is a highly prevalent neuro-cognitive complication of cirrhosis characterized by cognitive dysfunction, and high rate of subsequent mortality and recurrence[1]. Even after recovery from clinically evident episodes of OHE, most patients remain cognitively impaired with minimal hepatic encephalopathy (MHE)[2] and may not regain their pre-OHE cognitive function despite maximal therapy with the current standard of care[3, 4]. This cognitive impairment translates into poor health-related quality of life (HRQOL), loss of employment, and decline in socio-economic status [5, 6], which places a heavy burden on patients, caregivers, and society[3, 6, 7]. There is a major need for treating this continued cognitive impairment or MHE, but FDA-approved options are limited.

There is strong evidence that this persistent cognitive impairment is accompanied by a sustained pro-inflammatory and endothelial dysfunctional state that is not quenched by our current standard of care[8]. Ammonia, inflammation, oxidative stress, and endothelial dysfunction play an important role in the pathogenesis of OHE[9, 10]. There is also evidence that in patients with advanced cirrhosis there is a reduction in albumin concentration and capacity to bind metabolites that precipitate OHE[11], and this is the population at risk for developing OHE. During the inpatient phase, treating HE with intravenous (IV) albumin has shown conflicting results. [12, 13]

MHE and prior OHE impairs HRQOL without increasing the priority for transplant, therefore, in patients with relatively lower MELD, this can compound patient suffering. Thus, methods for treating persistent cognitive impairment in patients with MHE are needed.

### SUMMARY

It has been demonstrated herein that IV albumin significantly improves cognitive functioning and quality of life through reduction in endothelial dysfunction and systemic inflammation in cirrhotic patients who continue to have post-OHE cognitive dysfunction or MHE despite being on the current standard of care therapy.

Provided herein are methods of treating cognitive impairment in a subject suffering from cirrhosis, comprising administering to the subject a therapeutically effective amount of albumin, wherein the subject does not suffer from OHE. In some embodiments, the subject has not suffered from OHE within one month of the administering step. In some embodiments, the subject has MHE. In some embodiments, the subject has previously suffered from OHE. In some embodiments, the subject has not previously suffered from OHE. In some embodiments, the cognitive impairment is resistant to treatment with lactulose or rifaximin. In some embodiments, the albumin is administered intravenously. In some embodiments, the albumin is administered weekly for 3-7 weeks. In some embodiments, the therapeutically effective amount is 0.5-2 g/kg of body weight. In some embodiments, the subject does not have cognitive impairment caused by aging or an aging-associated disorder. In some embodiments, the subject does not have dementia.

Additional features and advantages of the present invention will be set forth in the description of disclosure that follows, and in part, will be apparent from the description of may be learned by practice of the disclosure. The disclosure will be realized and attained by the compositions and methods particularly pointed out in the written description and claims hereof.

### BRIEF DESCRIPTION OF THE DRAWINGS

FIG. 1. Overview of the study activities.

FIGS. 2A-B. PHES Change compared to baseline and end of drug (EOD). (A) Individual values show improvement (higher PHES) Score vs baseline in albumin but not placebo (N=24 per group). (B) Delta PHES (end of drug minus baseline) was greater in Albumin vs placebo (p=0.03, Mann-Whitney test). PHES: psychometric hepatic encephalopathy score, higher is better.

FIGS. 3A-C. End of Drug (EOD) Change in Minimal Hepatic Encephalopathy and Overall Improvement in Testing for PHES, Stroop and CFF. (A) Reversal of MHE Albumin group with PHES, Stroop, and CFF showed a significantly higher reversal in MHE using any test (all), PHES and Stroop post-albumin versus baseline but not with CFF. (B) Reversal of MHE Placebo group with any test (all), PHES, Stroop, and CFF showed no change in proportion with MHE at end of placebo versus baseline. (C) Proportion of individuals who experienced improvement in PHES and Stroop and trend towards CFF improvement was higher in albumin versus placebo at the end of drug. PHES: psychometric hepatic encephalopathy score, CFF: critical flicker frequency, MHE: minimal hepatic encephalopathy.

FIGS. 4A-I: Change in Sickness Impact Profile (SIP): higher-worse and has total, psychosocial and physical scores. (A, B, C) Total SIP, (D, E, F) Psychosocial SIP, (G, H, I) Physical SIP. Total and Psychosocial SIP improved after Albumin but not after placebo. Change in these parameters vs baseline was higher in Albumin group. No change in physical SIP was seen.**p<0.01, ***p<0.001, Ns: not significant.

### DETAILED DESCRIPTION

Embodiments of the present disclosure are directed toward methods for treating cognitive impairment in cirrhosis patients by administering albumin. Such treatment improves cognitive functioning and quality of life by reducing endothelial dysfunction and systemic inflammation.

Albumin, also referred to as serum albumin (e.g. human serum albumin) or blood albumin, is a globular protein found in vertebrate blood. It is produced in the liver and acts as a carrier protein for steroids, fatty acids, and thyroid hormones. Albumin for intravenous administration is commercially available, e.g. under the trade names Flexbumin®, Albuminar®, Alba®, Albuked®, Albutein®, Kedbumin®, Plasbumin®, and Albuminex®. The albumin solution is generally available at either 50 mg/ml (5%) or 250 mg/ml (25%). Suitable solutions may contain 25-350 mg/ml albumin.

The term “intravenous formulation,” refers to a single dose formulation of albumin that is provided as a lyophilized powder (or other solid form) that, once reconstituted in solution, is physiologically compatible with intravenous administration (e.g., by injection, infusion or otherwise). Alternatively, the terms refer to a formulation that is provided as a solution.

Cirrhosis is severe scarring of the liver which may be caused by many liver diseases and conditions such as hepatitis or chronic alcoholism. Hepatic encephalopathy encompasses a broad range of neuro-psychiatric disturbances that may accompany portosystemic shunting, acute liver failure, and cirrhosis. Cirrhotic encephalopathy is broadly classified as overt (OHE) and “minimal” or “covert” hepatic encephalopathy (MHE or CHE). MHE/CHE refers to the condition of that subset of patients with cirrhosis who do not have any clinically detectable neurologic abnormality but have abnormal neuropsychometric or neurophysiologic test results. Embodiments of the disclosure encompass treatment of cirrhotic subjects who have or have not previously suffered from OHE and may currently suffer from MHE/CHE or other cognitive impairment not due to OHE. In some embodiments, the subject has not suffered from OHE within one month from treatment.

Cognitive impairment in the absence of confusion or coma i.e., OHE (e.g. MHE) may be diagnosed using any one of three testing strategies known in the art, including the Psychometric hepatic encephalopathy score (PHES), Stroop test (e.g. the EncephalApp Stroop test), and Critical Flicker Frequency.

PHES is the score of the porto-systemic encephalopathy (PSE) battery, which is made up of five psychometrical tests. These include Digit Symbol Test (DST), Number connection Test-A (NCT-A), Number connection Test-B (NCT-B), Serial Dotting Test (SDT), and Line tracing Test (LTT). They assess psychomotor retardation, attention deficit and executive functions. The PHES is expressed as the number of standard deviation (SD) in a population matched with respect to age and educational level. In some embodiments, the subject to be treated has a PHES aggregate score of less than or equal to −4SD, e.g. a score of −15SD to −4SD.

The Stroop test measures the delay in reaction time between congruent and incongruent stimuli. The Stroop effect occurs when there is a mismatch between the name of a color and the color it is printed in. Stimuli in Stroop paradigms can be divided into three groups: neutral, congruent and incongruent. Neutral stimuli are those stimuli in which only the text, or color are displayed. Congruent stimuli are those in which the ink color and the word refer to the same color. Incongruent stimuli are those in which ink color and word differ. In some embodiments, the Stroop tests contains only neutral and incongruent stimuli. OffTime and OnTime seconds according to age, sex, and education matching norms are used to determine a positive or negative result for MHE. Such tests are known in the art and are available, e.g. at encephalapp.com.

The Critical Flicker Frequency (CFF) test measures the frequency at which a flickering light is perceived as continuous. In some embodiments, the subject to be treated has a CFF score of less than or equal to 39 Hz, e.g. 0-39 Hz.

The treatments that are undertaken reduce, reverse, slow (delay) the onset or progression of, or lessen the severity of, at least one symptom of cognitive dysfunction/impairment, and/or prevent, slow (delay) the onset or progression of, or lessen the severity of at least one symptom of cognitive dysfunction. For example, the treatment methods may prevent the progression to MHE/CHE, or the progression of MHE/CHE to OHE, and may in fact reverse symptoms of MHE/CHE.

Symptoms of MHE/CHE include, but are not limited to, difficulty with cognitive abilities such as executive decision-making and psychomotor speed; abnormalities in psychometric testing and slow response time; etc. In MHE/CHE, there are no clinical signs or symptoms of OHE; however, patients have neuropsychological deficiencies that can be detected with psychometric or neuropsychological testing. Unfortunately, patients with MHE/CHE tend to have poor quality of life, diminished work productivity, and increased traffic violations and accidents. Any of these symptoms can be reduced, avoided, or reversed by the methods described herein. For example, one or more of executive decision-making ability, psychomotor speed and performance in psychometric testing can be avoided or improved and, in some cases, a subject with MHE/CHE can revert to normal performance.

In OHE, there is a wide spectrum of symptomatic presentation and using the methods described herein one or more of the following symptoms can be avoided: grade symptoms include increased fatigue, poor short-term memory and concentration, and insomnia; grade 2 symptoms include confusion, changes in personality, bizarre behavior, and slurred speech; grade 3 symptoms include stupor; and grade 4 is characterized by coma, either responsive or unresponsive to noxious stimuli.

As used herein, the phrase “reduces a symptom” refers to reducing at least one of the frequency and amplitude of a symptom of a condition in a patient. In certain embodiments the patient enters remission and no longer experiences the symptom.

In some embodiments, the treatment described herein improves the quality of life of the subject. The term “quality of life” denotes the subjective feeling of well-being of a patient. The quality of life of a patient increases with reduction in or improvement of symptoms. The term “quality of life” is meant to define the collection of symptoms associated with cirrhosis without OHE, e.g. MHE. The quality of life may be measured using a Sickness Impact Profile (SIP). The SIP is a questionnaire which measured the physical and psychosocial domain. The overall maximum score for this test is 100% where a zero represents a good health status without physical or behavioral changes due to illness, while the 100 represents a poor health status or a major impact of illness on behavior. In some embodiments, the treatment described herein provides a significant improvement in quality of life based on the SIP measurement.

In some embodiments, the subject has experienced prior OHE. In some embodiments, the subject has experienced OHE more than one month prior to the treatment. One month may mean 28-31 days, e.g. about 30 days.

In some embodiments, the subject suffering from cognitive impairment without OHE (e.g. MHE) is resistant to current standard of care treatments such as lactulose or rifaximin, i.e. the subject does not respond or does not respond sufficiently to such treatments. For example, a resistant subject may not show significant improvement on any one of the PHES, Stroop, or CFF tests after treatment with lactulose or rifaximin. In some embodiments, the resistant subject may not have a significant improvement in quality of life, e.g. as measured by SIP, after treatment with lactulose or rifaximin.

In preferred embodiments, the cognitive impairment being treated is not caused by dementia, aging, or disorders associated with aging such as Alzheimer's disease, Parkinson's disease, or any form of dementia and the subject is not currently being treated for such disorders.

In some embodiments, the subject has a Mini-Mental State Examination (MMSE) score of less than or equal to 25. The MMSE is a questionnaire used to screen for cognitive impairment due to Alzheimer's disease or dementia.

The albumin treatment described herein may be administered concomitantly or sequentially, e.g. within the same hour or day, with one or more additional therapies, such as rifaximin, neomycin, metronidazole, lactulose, lactilol, lactose, ornithine aspartate, sodium benzoate, phenylacetate, probiotics, prebiotics, synbiotics, postbiotics, organ transplants, calcineurin inhibitors (CNIs) such as cyclosporine and tacrolimus, fecal transplants, administration of fecal matter from healthy donors, treatment for hepatitis B and hepatitis C, treatments for diabetes, hypertension and other related medical conditions, detoxification programs, and immunological therapies.

In other aspects, and/or in addition, life-style changes are prescribed, generally in combination with a medication. Such changes include but are not limited to: increased exercise; weight loss; consuming a brain-supportive diet (such as consumption of oily fish; omega 3 fatty acid supplements; cacao; berries; nuts; seeds; fruits; whole grains; coffee; tea; avocados; peanuts; leafy green vegetables; beans; lentils; herbs and spices with high antioxidant levels; vegetables high in fiber and inulin; fermented foods such as kimchi, sauerkraut, yogurts, kefir; and avoiding red meat, butter, high-fat dairy products and alcohol); increasing social connections; learning new tasks, etc.

In some embodiments, the administration is intravenous, oral, topical, parenteral, enteral, transdcrmal, intradermal, intraocular, intravitreal, sublingual, or intravaginal.

A patient or subject to be treated by any of the methods of the present disclosure can mean either a human or a non-human animal including, but not limited to dogs, horses, cats, rabbits, gerbils, hamsters, rodents, birds, aquatic mammals, cattle, pigs, camelids, and other zoological animals.

An albumin-containing formulation may comprise one or more pharmaceutically acceptable carriers. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

Suitable pharmaceutically acceptable carriers include but are not limited to water, salt solutions, alcohols, gum arabic, vegetable oils, benzyl alcohols, polyethylene glycols, gelate, carbohydrates such as lactose, amylose or starch, magnesium stearate talc, silicic acid, viscous paraffin, perfume oil, fatty acid monoglycerides and diglycerides, pentaerythritol fatty acid esters, hydroxymethylcellulose, polyvinylpyrrolidone, etc. The pharmaceutical preparations can be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. Other suitable excipients include, for example an inert diluent such as lactose; granulating and disintegrating agents such as cornstarch; binding agents such as starch; and lubricating agents such as magnesium stearate.

In some embodiments, the albumin formulation is administered to the subject in a therapeutically effective amount. By a “therapeutically effective amount” is meant a sufficient amount of active agent to treat the disease or disorder at a reasonable benefit/risk ratio applicable to any medical treatment. It will be understood that the total daily usage of the compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific active agent employed; and like factors well known in the medical arts. For example, it is well within the skill of the art to start doses of the compound at levels or frequencies lower than those required to achieve the desired therapeutic effect and to gradually increase the dosage or frequency until the desired effect is achieved. An effective amount of the drug is ordinarily supplied at a dosage level from 0.5 g/kg to about 2.5 g/kg of body weight, e.g. about 1.3-1.7 g/kg. In some embodiments, the composition is administered daily or 1, 2, 3, 4, 5, 6, 7, or more times weekly. In some embodiments, the composition is administered once weekly for 3-7 weeks, e.g. about 4-6 weeks. In some embodiments, the composition is administered once every 2 weeks, once every 3 weeks, or once every 4 weeks. In some embodiments, the administration continues for 2, 3, 4, 5, 6, 7, 8, 9, 10, 11, or 12 months or more.

Depending on the concentration of albumin in an intravenous formulation solution and consistent with the dose levels described herein, the volume of intravenous formulation solution to be administered can vary from about 1 mL to about 200 mL, e.g. about 50-100 mL. In some embodiments, the amount of time required for administration of the intravenous formulation ranges from about 1 minute to about 2 hours, e.g. about 45 to 75 minutes.

It is to be understood that this invention is not limited to any particular embodiment described herein and may vary. It is also to be understood that the terminology used herein is for the purpose of describing particular embodiments only, and is not intended to be limiting, since the scope of the present invention will be limited only by the appended claims.

Where a range of values is provided, it is understood that each intervening value between the upper and lower limit of that range (to a tenth of the unit of the lower limit) is included in the range and encompassed within the invention, unless the context or description clearly dictates otherwise. In addition, smaller ranges between any two values in the range are encompassed, unless the context or description clearly indicates otherwise.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%.

Unless defined otherwise, all technical and scientific terms used herein have the same meaning as commonly understood by one of ordinary skill in the art to which this invention belongs. Representative illustrative methods and materials are herein described; methods and materials similar or equivalent to those described herein can also be used in the practice or testing of the present invention.

All publications and patents cited in this specification are herein incorporated by reference as if each individual publication or patent were specifically and individually indicated to be incorporated by reference, and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual dates of public availability and may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as support for the recitation in the claims of such exclusive terminology as “solely,” “only” and the like in connection with the recitation of claim elements, or use of a “negative” limitations, such as “wherein [a particular feature or element] is absent”, or “except for [a particular feature or element]”, or “wherein [a particular feature or element] is not present (included, etc.) . . . ”.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order, which is logically possible.

### Example

### SUMMARY

Even after recovery from overt hepatic encephalopathy (HE), minimal HE (MHE), which impairs quality of life (QOL), can persist. Treatment options are limited. Our aim was to determine the impact of albumin versus saline on MHE and QOL in patients with prior HE already on standard of care using double-blind, placebo-controlled randomized clinical trial. Outpatients with cirrhosis and prior HE, MHE and hypoalbuminemia already on HE-treatment were included. Patients on regular IV albumin infusions were excluded. Subjects were randomized 1:1 to receive either weekly infusions of 25% IV albumin 1.5 g/kg or saline over 5 weeks (end-of-drug, EOD) and then 1-week post-infusion (end-of-study, EOS). MHE was defined using either Psychometric hepatic encephalopathy score (PHES), Stroop or Critical clicker frequency. MHE and QOL using Sickness Impact profile (SIP total, physical, psychosocial domain, higher=worse) and serum (inflammation, endothelial dysfunction, and ischemia-modified albumin IMA) were compared between baseline, EOD and EOS. 48(24/group) subjects were randomized and were balanced at baseline, including HE-medication use. Adverse events were similar, with MELD and ammonia remaining stable between/within groups. Albumin levels increased and IMA decreased only in the albumin group at EOD and EOS vs baseline. PHES and Stroop MHE reversal and improvement was greater in albumin group at EOD and persisted at EOS. SIP total and psychosocial, but not physical domain improved in the albumin but not placebo group versus baseline at EOD and EOS along with significant reduction in IL-1β, and endothelial dysfunction markers. To conclude, in a double-blind, placebo controlled RCT of outpatients with cirrhosis, prior HE and current MHE, albumin infusions were associated with improved cognitive function and psychosocial quality of life likely through amelioration of endothelial dysfunction.

### Methods

After informed consent, we included patients >18 years with cirrhosis diagnosed using the following criteria: liver biopsy, transient wave elastography (>20 KPa) or radiological evidence consistent with cirrhosis. Also, if a patient with chronic liver disease had endoscopic or radiological evidence of varices, or platelet count <150,000/mm3 and AST/ALT ratio >1, they were included.

We required patients to have experienced prior overt HE with West-Haven grade 0 on lactulose or rifaximin for at least 2 months prior to enrollment and have a serum albumin of <4 gm/dl who had MHE (details below). Exclusion criteria included no prior HE, those with higher West-Haven grades despite therapy at the time of enrollment, those who recently received albumin infusions in the past 3 months for any cause or were anticipated to do so during the study period, recent alcohol or illicit drug use (<3 months), infections (<1 month), allergies to albumin, congestive heart failure, those unable to consent, and those unlikely to survive 6 weeks in the opinion of the principal investigator (PI). Alcohol use was assessed using AUDIT-10 questionnaires and anyone with >8 was excluded. Direct questioning was used to evaluate for alcohol misuse during each visit.

After consent, screening procedures included psychometric tests, pregnancy testing, and mini-mental status. Lack of cognitive impairment on any of the criteria, Mini-Mental State Examination (MMSE) score <25, or pregnancy were exclusions after screening.

Cognitive impairment on any of the three testing strategies for MHE, including Psychometric hepatic encephalopathy score (PHES). Stroop test and Critical Flicker Frequency (PHES aggregate score <−4SD or Stroop OffTime+OnTime based on norms or CFF <39 Hz) on screening qualified patients for the trial[2, 14-16].

HRQOL: We administered the Sickness Impact Profile (SIP), a validated 136 questions HRQOL scale which yields an overall score, a physical score, and a psychosocial score[17]; a high score indicates poor HRQOL.

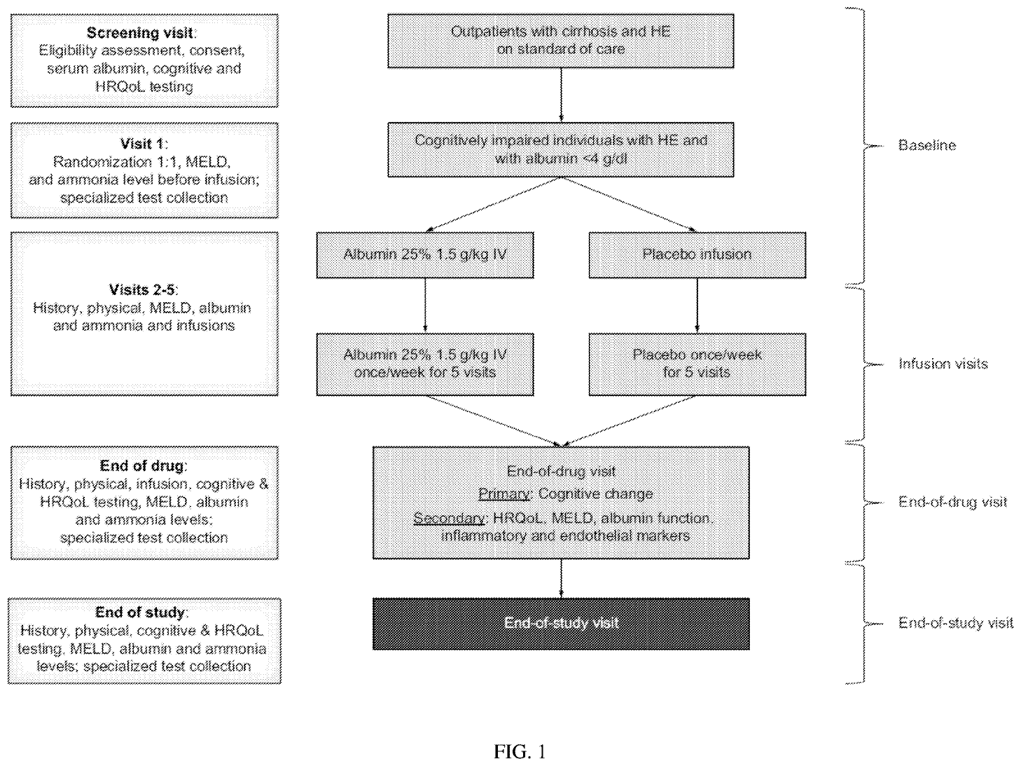

An overview of the RCT is in FIG. 1. The trial was registered at clinicaltrials.gov NCT03585257 before enrollment of the first patient.

Randomization: 1:1 randomization was performed in blocks of 4 using a random number generator created by the Richmond VA Investigational pharmacy.

Blinding: All infusion tubing was covered in foil. Since albumin levels were not allowed to go over 4 g/dl, an albumin level was checked before the infusion. The outcomes assessor and patients were blinded to the albumin results. This was only broken for a serious adverse event (SAE) if there was a reasonable suspicion that it was related to the use of albumin.

Albumin dosing: Intravenous 25% albumin infusion 1.5 g/kg body weight (maximal dose given was limited to 100 gm) versus placebo infusion (equivalent amount of normal saline) was administered once a week for five weeks for a maximum of 5 infusions over sixty minutes per clinical treatment protocol. Pre-infusion serum albumin was checked by the pharmacy, and if >4.0 gm/dl, then normal saline was given instead, if patient was assigned to the albumin group. The total number of grams of albumin infused over the 5 weeks was calculated and compared between groups. The study protocol was approved by the Richmond VA IRB and carried out under FDA IND.

### Analytic Plan and Outcomes

Primary: Change in cognitive function in albumin group compared to the placebo group on PHES at baseline compared to end-of-treatment visit.

Secondary: (a) Change in HRQOL using Sickness Impact Profile (b) Changes on other cognitive tests, (c) Change in MELD score, (d) development of new complications of cirrhosis, infections, or hospitalizations during the study, (e) changes in specialized blood markers for inflammation, endotoxemia, endothelial dysfunction and albumin function, (f) adverse events and discontinuation related to IV albumin infusion and (g) changes to these parameters after albumin infusion was stopped for a week.

Exploratory with specialized blood work: We also conducted a biochemical analysis on serum samples drawn at baseline and after infusions and end of study for % ischemia-modified albumin (IMA), serum lipopolysaccharide-binding protein (LBP), inflammatory cytokines (IL-6, TNF-α, IL-1β, IL-1β), and markers of endothelial dysfunction endothelial dysfunction (Asymmetric Dimethyl Arginine (ADMA) and soluble intercellular adhesion molecule (sICAM-1))[8].

Sample size: In a prior study of patients without HE, using rifaximin, there was a 90% improvement in the treatment group compared to 60% with placebo and in another study using fecal transplant in HE, there was again a 90% improvement compared to standard of care[18, 19]. Since there was significantly higher cognitive impairment in our group with prior HE, we expected the placebo not to respond as well and therefore, assumed that 70% of the albumin group would improve compared to 30% of the placebo group. Based on this assumption, 24 patients were required in each group, resulting in a total of 48 patients.

Statistical plan: We performed a pre vs post analysis for end of study drug (EOD) values for cognitive testing, safety laboratories, and SIP within groups using paired t-tests or Wilcoxon signed rank tests. The proportion with improvement in cognitive tests, and those with MHE reversal between groups were compared using Chi-square testing. Delta PHES, and SIP between groups (EOD minus baseline) were compared between groups using Mann Whitney tests. A repeated measures analysis was performed for PHES scores using time and group interaction analysis from baseline to EOD.

P<0.05 was considered significant. In case patients missed appointments or withdrew, the last value was carried forward and an intention to treat analysis was performed. We also performed an end-of-study (EOS) comparison for values 1 week after the completion of infusions using RMANOVA within groups.

### Results

Baseline comparisons: As shown in Table 1, both groups were similar with respect to demographics, cirrhosis and HE-related factors and prior complications, cognitive testing, other medications including lactulose, rifaximin, beta-blockers, diuretics and statins, and laboratory values.

TABLE 1
Baseline Measures between groups

| | Placebo Group | Albumin Group | |
|---|---|---|---|
| | (n = 24) | (n = 24) | P value |
| Age | 62.21 ± 8.59 | 63.83 ± 6.99 | 0.48 |
| Male Gender | 21 (88%) | 22 (92%) | 1.00 |
| Caucasian | 16 (67%) | 19 (79%) | 0.49 |
| Education level | 14.17 ± 2.24 | 13.04 ± 2.74 | 0.13 |
| BMI (kg/m2) | 34.82 ± 6.45 | 33.32 ± 5.37 | 0.39 |
| Cirrhosis details | | | |
| Etiology (HCV/alcohol/ | 4/10/10/0 | 3/12/7/2 | 0.41 |
| NASH/others) | | | |
| Prior Ascites | 15 (63%) | 15 (63%) | 1.0 |
| Prior variceal bleed | 5 (20%) | 7 (28%) | 0.50 |
| Non-selective BB | 15 (63%) | 14 (58%) | 0.77 |
| Diuretics | 15 (63%) | 14 (58%) | 0.77 |
| Statin | 6 (25%) | 4 (4%) | 0.72 |
| Benzodiazepines | 0 (0%) | 2 (8%) | 0.45 |
| Lactulose | 21 (88%) | 20 (83%) | 1.0 |
| Rifaximin | 20 (83%) | 21 (88%) | 1.0 |
| Lab values | | | |
| MELD-Na score | 10.46 ± 3.36 | 11.75 ± 3.78 | 0.22 |
| Platelet count | 113.2 ± 73.2 | 96.4 ± 55.1 | 0.37 |
| Serum Na | 138.8 ± 2.5 | 138.9 ± 2.2 | 0.85 |
| Serum albumin | 3.20 ± 0.38 | 3.38 ± 0.36 | 0.20 |
| Venous ammonia | 83.0 ± 40.4 | 75.5 ± 44.6 | 0.54 |
| Serum bilirubin | 1.32 ± 0.75 | 1.24 ± 0.75 | 0.72 |
| INR | 1.27 ± 0.16 | 1.27 ± 0.19 | 1.00 |
| Serum creatinine | 0.88 ± 0.27 | 1.18 ± 0.50 | 0.01 |
| Cognitive testing | | | |
| PHES total | −5.04 ± 3.85 | −6.00 ± 5.08 | 0.47 |
| NCT-A (seconds) | 54.3 ± 23.3 | 59.8 ± 30.7 | 0.49 |
| NCT-B (seconds) | 122.2 ± 66.3 | 145.0 ± 86.5 | 0.32 |
| Digit symbol | 42.8 ± 13.7 | 38.3 ± 10.7 | 0.22 |
| Serial dotting | 80.9 ± 23.7 | 95.3 ± 44.7 | 0.17 |
| Line tracing errors | 34.5 ± 21.9 | 46.1 ± 23.8 | 0.09 |
| Line tracing time | 99.3 ± 40.3 | 94.8 ± 33.2 | 0.68 |
| (seconds) | | | |
| EncephalApp | | | |
| OffTime (seconds) | 98.9 ± 18.9 | 104.7 ± 32.6 | 0.45 |
| OnTime (seconds) | 126.6 ± 37.9 | 130.6 ± 63.1 | 0.79 |
| Off run number | 5.38 ± 0.88 | 5.71 ± 0.75 | 0.16 |
| On runs number | 6.83 ± 3.43 | 6.67 ± 1.90 | 0.84 |
| Off + On time (time) | 220.8 ± 56.8 | 235.3 ± 91.4 | 0.51 |
| Critical flicker frequency (Hz) | 39.74 ± 4.17 | 39.93 ± 5.62 | 0.90 |
| MHE frequency | | | |
| MHE on PHES | 14 (58%) | 15 (63%) | 1.0 |
| MHE on EncephalApp | 23 (96%) | 23 (96%) | 0.61 |
| MHE on CFF | 11 (46%) | 9 (38%) | 0.78 |
| Sickness Impact Profile | | | |
| Total Score | 17.7 ± 10.4 | 23.3 ± 13.8 | 0.62 |
| Psychosocial domain | 17.3 ± 12.2 | 23.4 ± 15.3 | 0.12 |
| Physical domain | 13.7 ± 10.6 | 17.7 ± 13.2 | 0.26 |

Clinical course: One patient in the albumin group developed hives 50 minutes after the first infusion, which led to unblinding and discontinuation. Two other patients assigned to albumin required hospitalizations during the study, one because of HE precipitated by dehydration and one due to exacerbation of a pre-existing foot ulcer. Both resumed their infusions. Four patients in the placebo group required hospitalizations. Three were admitted with infections: one severe UTI leading to HE, one with leg cellulitis, and one with spinal epidural abscess. The patients with UTI and cellulitis did not want to proceed, while the person with epidural abscess developed it on the last day of the study. One patient was admitted for an elective HCC ablation, after which he continued with the study. Apart from the allergic reaction, none of the other patients' assignments were unblinded.

No changes in MELD score, or serum sodium were seen in either group. As expected, albumin levels were significantly higher at end-of-drug compared to baseline in only the treatment group (Table 2). None of the subjects showed evidence of alcohol misuse during the study and no changes in medications from baseline that could affect cirrhosis progression (beta-blockers, diuretics, HE-medications, or stains) were seen.

TABLE 2
Comparisons between Baseline and End of Drug within groups

| | Placebo | Placebo | P value | Albumin | Albumin | P value |
|---|---|---|---|---|---|---|
| | Baseline | End Drug | (paired) | Baseline | End Drug | (paired) |
| Lab values | | | | | | |
| MELD score | 10.46 ± 3.36 | 10.58 ± 3.86 | 0.66 | 11.75 ± 3.78 | 12.46 ± 4.21 | 0.07 |
| Serum Na | 138.8 ± 2.5 | 138.75 ± 2.44 | 0.92 | 138.9 ± 2.2 | 138.9 ± 2.0 | 0.94 |
| Serum | 3.20 ± 0.38 | 3.19 ± 0.49 | 0.94 | 3.38 ± 0.36 | 3.81 ± 0.33 | <0.0001 |
| albumin | | | | | | |
| Venous | 83.0 ± 40.4 | 87.2 ± 48.5 | 0.65 | 75.5 ± 44.6 | 74.0 ± 41.8 | 0.75 |
| ammonia | | | | | | |
| Platelet | 113.2 ± 73.2 | 112.5 ± 72.7 | 0.98 | 96.4 ± 55.1 | 93.5 ± 51.5 | 0.86 |
| count | | | | | | |
| Cognitive | | | | | | |
| testing | | | | | | |
| PHES total | −5.04 ± 3.85 | −4.79 ± 4.22 | 0.62 | −6.00 ± 5.08 | −4.20 ± 4.20 | 0.009 |
| NCT-A | 54.3 ± 23.3 | 46.0 ± 16.5 | 0.01 | 59.8 ± 30.7 | 47.7 ± 22.3 | 0.004 |
| (seconds) | | | | | | |
| NCT-B | 122.2 ± 66.3 | 125.4 ± 59.5 | 0.79 | 145.0 ± 86.5 | 136.6 ± 93.5 | 0.47 |
| (seconds) | | | | | | |
| Digit symbol | 42.8 ± 13.7 | 45.0 ± 17.7 | 0.13 | 39.1 ± 10.1 | 40.9 ± 10.7 | 0.25 |
| Serial dotting | 80.9 ± 23.7 | 85.0 ± 41.1 | 0.54 | 95.3 ± 44.7 | 78.0 ± 41.4 | 0.01 |
| Line | 34.5 ± 21.9 | 36.6 ± 17.6 | 0.51 | 46.1 ± 23.8 | 48.5 ± 24.1 | 0.49 |
| tracing errors | | | | | | |
| Line tracing | 99.3 ± 40.3 | 91.8 ± 31.9 | 0.23 | 94.8 ± 33.2 | 78.5 ± 23.4 | 0.002 |
| time (seconds) | | | | | | |
| EncephalApp | | | | | | |
| OffTime | 98.9 ± 18.9 | 98.6 ± 20.6 | 0.78 | 104.7 ± 32.6 | 100.2 ± 23.3 | 0.50 |
| (seconds) | | | | | | |
| OnTime | 126.6 ± 37.9 | 122.5 ± 36.4 | 0.42 | 130.6 ± 63.1 | 129.7 ± 75.5 | 0.89 |
| (seconds) | | | | | | |
| Off run number | 5.38 ± 0.88 | 5.48 ± 0.79 | 0.62 | 5.71 ± 0.75 | 5.54 ± 0.72 | 0.42 |
| On runs number | 6.83 ± 3.43 | 5.83 ± 1.50 | 0.06 | 6.67 ± 1.90 | 5.88 ± 1.33 | 0.10 |
| Off + On | 220.8 ± 56.8 | 223.2 ± 54.4 | 0.96 | 235.3 ± 91.4 | 228.0 ± 91.6 | 0.32 |
| time (time) | | | | | | |
| Critical flicker | 39.74 ± 4.17 | 40.34 ± 4.29 | 0.44 | 39.93 ± 5.62 | 40.22 ± 4.80 | 0.78 |
| frequency (Hz) | | | | | | |
| MHE frequency | | | | | | |
| MHE on PHES | 14 (58%) | 13 (54%) | 1.0 | 15 (63%) | 7 (29%) | 0.015 |
| MHE on | 23 (96%) | 23 (96%) | 1.0 | 23 (96%) | 17 (71%) | 0.02 |
| EncephalApp | | | | | | |
| MHE on CFF | 11 (46%) | 11 (46%) | 1.0 | 9 (38%) | 6 (25%) | 0.30 |
| MHE (regardless | 24 (100%) | 23 (96%) | 0.95 | 24 (100%) | 17 (71%) | 0.01 |
| of instrument) | | | | | | |
| Sickness | | | | | | |
| Impact Profile | | | | | | |
| Total Score | 17.7 ± 10.4 | 16.0 ± 9.73 | 0.59 | 23.3 ± 13.8 | 17.8 ± 14.3 | 0.004 |
| Psychosocial | 17.3 ± 12.2 | 16.1 ± 11.4 | 0.86 | 23.4 ± 15.3 | 17.2 ± 20.2 | 0.005 |
| domain | | | | | | |
| Physical | 13.7 ± 10.6 | 11.3 ± 9.7 | 0.08 | 17.7 ± 13.2 | 16.2 ± 17.6 | 0.11 |
| domain | | | | | | |

The group assigned to albumin received a total of 312.92±110.74 grams of albumin during the visits. Six patients received albumin at all 5 visits, four received it at 4 visits, 12 received albumin during 3 visits, and two received 2 infusions; rest of the infusions were saline to prevent the serum albumin from going above 4.0 gm/dl. None of the patients received non-study albumin either during or after the 1-week post infusions in this trial or during hospitalizations.

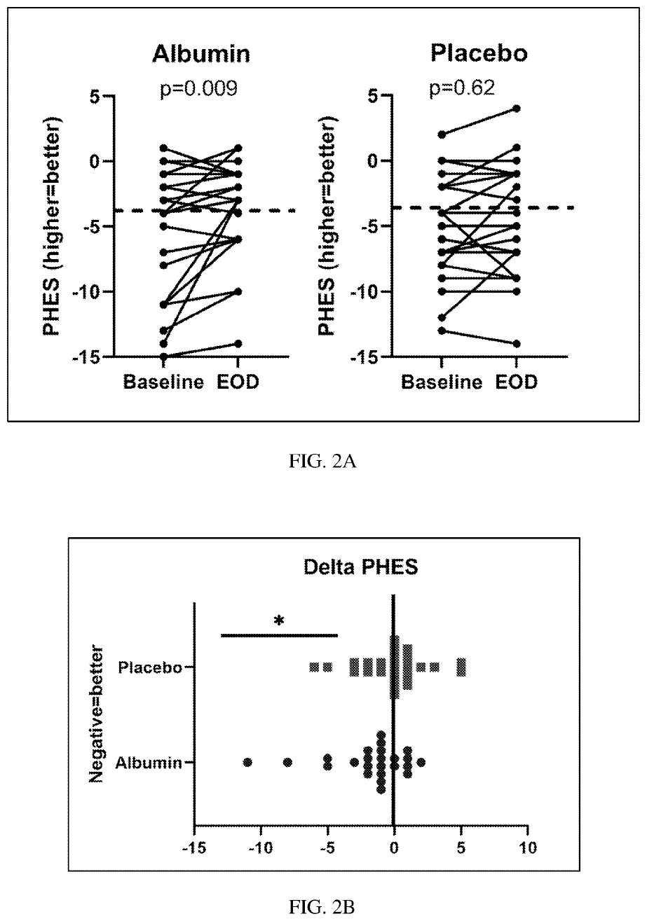

MHE and cognitive change: There was a significant improvement in overall PHES scores in the albumin but not the placebo-assigned groups, while a trend towards improvement in EncephalApp and CFF scores was seen in the treatment group only. Delta PHES was also higher in albumin vs placebo groups (FIG. 2, Table 2).

At baseline, most patients showed MHE on EncephalApp; the one patient in each group without MHE on EncephalApp was positive for MHE on CFF. All EncephalApp MHE positive patients were also positive for MHE on PHES.

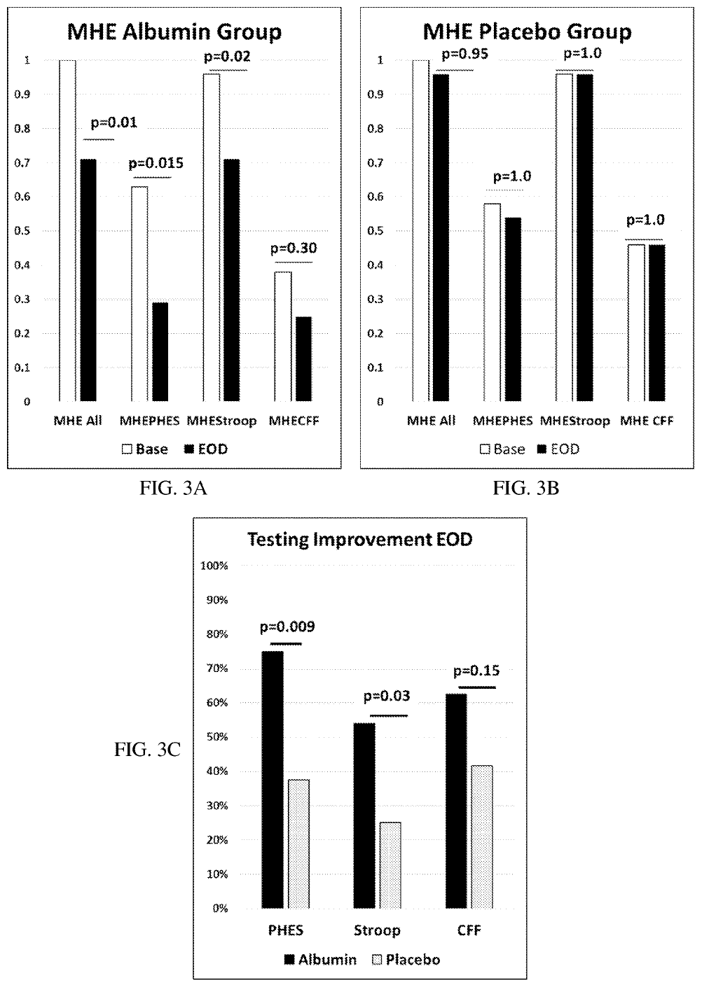

In the albumin group, there was a significantly lower MHE rate on PHES and EncephalApp compared to baseline but a trend towards reduction in MHE on CFF (FIG. 3A). Any MHE also reduced with albumin from 100% to 71%. In the placebo group, there was no significant change in MHE proportion on any modality or overall (FIG. 3B).

As shown in Table 2 and FIG. 3C, when only improvement in scores (not stability or worsening) was compared between groups from baseline to end-of-drug, there was a significantly higher proportion of patients in the albumin-assigned groups who improved their PHES and EncephalApp performance, with a trend towards higher improvement in CFF, compared to changes in the placebo-assigned group.

On repeated measures analysis, there was a statistically significant (p=0.04) interaction effect, which was attributable to the increase in PHES (improvement) in the albumin group (p=0.002) whereas there was no change in the placebo group (p=0.77). No similar changes were seen for Stroop or CFF.

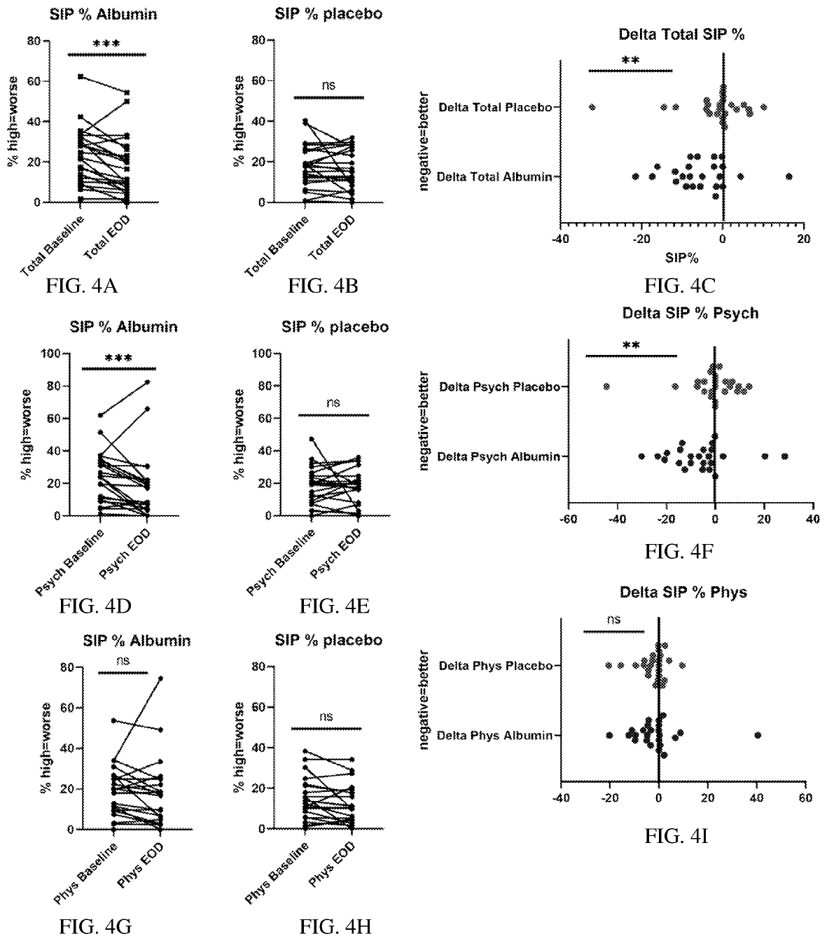

HRQOL: There was a significant improvement in SIP total and psychosocial compared to baseline in albumin but not the placebo group (FIG. 4). This was also accompanied by a higher delta SIP in these two parameters in the albumin vs placebo group. No significant trend in the physical domain of SIP was found.

Specialized blood work: No significant difference was seen between groups at baseline (Table 4). There was a significant reduction in IL-1p, ICAM-1, ADMA and IMA post-albumin compared to baseline, but no change was seen in the placebo group. No changes in the LBP or other inflammatory markers were seen (Table 4).

As end of study comparisons, we measured these variables one week after the final infusion (placebo or albumin). The results showed that serum albumin continued to increase in the albumin-assigned group and despite worsening of two patients with respect to their PHES and EncephalApp MHE status, the overall percentage with MHE remained significantly lower in albumin-assigned subjects compared to their baseline (Table 3). There was also a continued improvement in total and psychosocial SIP, which was accompanied by lower serum IL-1β, ADMA and IMA even at EOS. On the other hand, in the placebo group, there was a continued worsening of ICAM-1, IMA and IL-10 even at ES visits (Table 4). None of the individual cognitive tests, laboratory values including ammonia, or liver disease severity changed over time. There was no difference in cognitive performance, SIP values or specialized biomarkers based on the number of albumin infusions received, likely because the number of infusions were titrated towards the albumin levels.

TABLE 3
Changes in Clinical Parameters at EOD and EOS compared to Baseline

| | Placebo | Placebo | Placebo | P | Albumin | Albumin | Albumin | P |
|---|---|---|---|---|---|---|---|---|
| | Baseline | EOD | EOS | value | Baseline | EOD | EOS | value |
| Lab values | | | | | | | | |
| MELD score | 10.46 ± 3.36 | 10.58 ± 3.86 | 10.79 ± 3.64 | 0.59 | 11.75 ± 3.78 | 12.46 ± 4.21 | 12.75 ± 3.81 | 0.08 |
| Serum Na | 138.8 ± 2.5 | 138.75 ± 2.44 | 139.3 ± 3.47 | 0.23 | 138.9 ± 2.2 | 138.9 ± 2.0 | 138.8 ± 2.5 | 0.95 |
| Serum | 3.20 ± 0.38 | 3.19 ± 0.49 | 3.23 ± 0.47 | 0.79 | 3.38 ± 0.36 | 3.81 ± 0.33 | 3.93 ± 0.33 | <0.001 |
| albumin | | | | | | | | |
| Venous | 83.0 ± 40.4 | 87.2 ± 48.5 | 78.46 ± 41.78 | 0.42 | 75.5 ± 44.6 | 74.0 ± 41.8 | 64.81 ± 41.1 | 0.20 |
| ammonia | | | | | | | | |
| Cognitive | | | | | | | | |
| testing | | | | | | | | |
| PHES total | −5.04 ± 3.85 | −4.79 ± 4.22 | −4.63 ± 4.52 | 0.61 | −6.00 ± 5.08 | −4.20 ± 4.20 | −5.04 ± 4.70 | 0.012 |
| NCT-A | 54.3 ± 23.3 | 46.0 ± 16.5 | 46.1 ± 16.9 | 0.28 | 59.8 ± 30.7 | 47.7 ± 22.3 | 48.3 ± 21.3 | 0.18 |
| (seconds) | | | | | | | | |
| NCT-B | 122.2 ± 66.3 | 125.4 ± 59.5 | 124.2 ± 76.6 | 0.99 | 145.0 ± 86.5 | 136.6 ± 93.5 | 136.0 ± 82.0 | 0.22 |
| (seconds) | | | | | | | | |
| Digit symbol | 42.8 ± 13.7 | 45.0 ± 17.7 | 47.0 ± 17.6 | 0.68 | 39.1 ± 10.1 | 40.9 ± 10.7 | 38.8 ± 13.5 | 0.93 |
| (raw score) | | | | | | | | |
| Serial dotting | 80.9 ± 23.7 | 85.0 ± 41.1 | 85.9 ± 38.7 | 0.89 | 95.3 ± 44.7 | 78.0 ± 41.4 | 80.9 ± 37.9 | 0.31 |
| (raw score) | | | | | | | | |
| Line tracing | 34.5 ± 21.9 | 36.6 ± 17.6 | 91.5 ± 39.4 | 0.69 | 46.1 ± 23.8 | 48.5 ± 24.1 | 85.5 ± 32.1 | 0.17 |
| errors (number) | | | | | | | | |
| Line tracing | 99.3 ± 40.3 | 91.8 ± 31.9 | 38.4 ± 20.8 | 0.81 | 94.8 ± 33.2 | 78.5 ± 23.4 | 50.3 ± 21.2 | 0.83 |
| time(seconds) | | | | | | | | |
| EncephalApp | | | | | | | | |
| OffTime | 98.9 ± 18.9 | 98.6 ± 20.6 | 98.5 ± 26.2 | 0.99 | 104.7 ± 32.6 | 100.2 ± 23.3 | 97.2 ± 23.8 | 0.66 |
| (seconds) | | | | | | | | |
| OnTime | 126.6 ± 37.9 | 122.5 ± 36.4 | 118.7 ± 44.9 | 0.80 | 130.6 ± 63.1 | 129.7 ± 75.5 | 123.4 ± 68.3 | 0.93 |
| (seconds) | | | | | | | | |
| Off run number | 5.38 ± 0.88 | 5.48 ± 0.79 | 5.38 ± 0.74 | 0.92 | 5.71 ± 0.75 | 5.54 ± 0.72 | 5.60 ± 0.96 | 0.73 |
| On runs number | 6.83 ± 3.43 | 5.83 ± 1.50 | 6.62 ± 2.85 | 0.41 | 6.67 ± 1.90 | 5.88 ± 1.33 | 6.27 ± 2.03 | 0.35 |
| Off + On | 220.8 ± 56.8 | 223.2 ± 54.4 | 223.7 ± 64.2 | 0.99 | 235.3 ± 91.4 | 228.0 ± 91.6 | 219.3 ± 84.5 | 0.34 |
| time (time) | | | | | | | | |
| Critical flicker | 39.74 ± 4.17 | 40.34 ± 4.29 | 40.56 ± 4.60 | 0.45 | 39.93 ± 5.62 | 40.22 ± 4.80 | 41.51 ± 4.09 | 0.19 |
| frequency (Hz) | | | | | | | | |
| MHE frequency | | | | | | | | |
| MHE on PHES | 14 (58%) | 13 (54%) | 11 (46%) | 0.67 | 15 (63%) | 7 (29%) | 9 (38%) | 0.05 |
| MHE on | 23 (96%) | 23 (96%) | 23 (96%) | 1.0 | 23 (96%) | 17 (71%) | 19 (79%) | 0.07 |
| EncephalApp | | | | | | | | |
| MHE on CFF | 11 (46%) | 11 (46%) | 11 (46%) | 1.0 | 9 (38%) | 6 (25%) | 9 (38%) | 0.57 |
| MHE (any | 24 (100%) | 23 (96%) | 23 (96%) | 0.96 | 24 (100%) | 17 (71%) | 19 (79%) | 0.05 |
| strategy) | | | | | | | | |
| SIP | | | | | | | | |
| Total Score | 17.7 ± 10.4 | 16.0 ± 9.73 | 14.3 ± 11.0 | 0.09 | 23.3 ± 13.8 | 17.8 ± 14.3 | 17.1 ± 14.4 | 0.008 |
| Psychosocial | 17.3 ± 12.2 | 16.1 ± 11.4 | 13.3 ± 10.4 | 0.06 | 23.4 ± 15.3 | 17.2 ± 20.2 | 15.3 ± 16.9 | 0.004 |
| domain | | | | | | | | |
| Physical | 13.7 ± 10.6 | 11.3 ± 9.7 | 11.0 ± 14.1 | 0.22 | 17.7 ± 13.2 | 16.2 ± 17.6 | 13.2 ± 12.4 | 0.08 |
| domain | | | | | | | | |

Comparisons using RMANOVA within groups, significant comparisons are in bold text

TABLE 4
Changes in Specialized Blood Markers over the Trial

| | Placebo | Placebo | Placebo | Albumin | Albumin | Albumin |
|---|---|---|---|---|---|---|
| | Baseline | EOD | EOS | Baseline | EOD | EOS |
| IL-1β | 0.53 ± 0.57 | 0.50 ± 0.47 | 0.47 ± 0.50 | 0.42 ± 0.39 | 0.37 ± 0.29* | 0.35 ± 0.37* |
| (pg/ml) | | | | | | |
| IL-6 | 3.61 ± 2.67 | 3.80 ± 2.23 | 4.94 ± 7.52 | 3.71 ± 2.56 | 3.91 ± 2.61 | 3.18 ± 1.73 |
| (pg/ml) | | | | | | |
| TNFα | 15.55 ± 8.34 | 15.09 ± 6.23 | 16.88 ± 7.32 | 16.34 ± 14.46 | 14.46 ± 7.06 | 15.06 ± 7.94 |
| (pg/ml) | | | | | | |
| IL-10 | 3.69 ± 3.09 | 2.93 ± 3.17* | 3.08 ± 3.02* | 4.01 ± 4.07 | 3.83 ± 2.96 | 3.28 ± 1.81 |
| (pg/ml) | | | | | | |
| LBP | 1784.9 ± 1557.3 | 1714.8 ± 1255.5 | 1931.2 ± 316.7 | 1651.1 ± 952.1 | 1669.8 ± 1010.4 | 1659.7 ± 931.6 |
| (ng/ml) | | | | | | |
| ICAM-1 | 298.1 ± 97.1 | 341.6 ± 118.8* | 343.6 ± 125.9* | 316.7 ± 140.3 | 271.1 ± 134.1*^{†} | 313.1 ± 125.6 |
| (ng/ml) | | | | | | |
| ADMA | 0.65 ± 0.12 | 0.72 ± 0.13* | 0.65 ± 0.14 | 0.69 ± 0.13 | 0.63 ± 0.09*^{†} | 0.63 ± 0.10* |
| (uM) | | | | | | |
| IMA | 831.7 ± 1335.6 | 997.2 ± 1529.3* | 1604.9 ± 3082.3* | 1491.5 ± 3125.9 | 1144.1 ± 2812.6* | 1042.9 ± 2753.9* |
| (IU/ml) | | | | | | |

EOD: end of drug, EOS: end of study.
*p < 0.05 compared to baseline (Wilcoxon Signed rank test and paired t-tests),
^{†}p < 0.05 compared to placebo (Kruskal-Wallis test). No significant difference between groups at baseline.

### Discussion

The current randomized trial shows intravenous albumin is associated with improvement in cognitive dysfunction and psychological quality of life in patients with cirrhosis with prior HE. There was an improvement in specialized blood markers focusing on inflammation, endothelial dysfunction, and albumin function in the albumin group compared to the placebo group.

There is a major need to improve cognition in patients with prior HE since this translates into clinical and psychosocial burden felt by patients, families, and the healthcare systemns[20, 21]. In the US, apart from lactulose and rifaximin, there are no other FDA-approved therapies for this condition, which is often under-treated and under-recognized[2]. In this trial, we focused on this under-served population and used IV albumin, to both improve clinical outcomes and further explore the reduction of inflammation and endothelial dysfunction [22].

In the trial, a pragmatic definition of MHE was employed that utilized any of the three recommended tests due to the differing parts of the brain that are interrogated by these cognitive tests[1]. We found that the majority of subjects were impaired on EncephalApp or PHES, which test similar parts of the brain, and ultimately responded to albumin to a greater extent than CFF[2]. The results demonstrated a better cognitive performance after albumin compared to baseline, but not with placebo with respect to any improvement in test performance, proportion of patients with any improvement, or MHE reversal. Moreover, the delta PHES was significantly improved in the albumin compared to placebo group. The results also confirm prior study results where the learning capability of patients after HE was diminished since there was no change in performance after placebo therapy[3, 23, 24], which makes the improvement in the albumin group even more striking.

More importantly, the delta PHES was accompanied by an improvement in the psychosocial aspect of the SIP that would translate improved cognitive performance into improved daily functioning. The improvement in psychosocial but not physical aspects of the SIP points towards a selective betterment of the personal interpretation of brain function that accompanied the objective cognitive enhancement. Since impaired daily function is linked with high family burden and worse socio-economic status in HE, HRQOL improvement is an important and impactful finding[20, 21, 25].

While the serum albumin levels increased as expected, our specialized blood work showed that the impaired function of albumin detected by IMA was reduced in the albumin group and increased in the placebo group. This was also accompanied by alterations in cytokines (reduced inflammatory IL-13 in the albumin group and decreased anti-inflammatory IL-10 in the placebo group) as well as reduction in endothelial dysfunction markers, ADAM, ICAM-1, in the albumin group. This was despite including the baseline values as last observation carried forward for two infected placebo patients. There was importantly no change in liver disease severity or venous ammonia levels between and within groups. This could explain the greater improvement in PHES that is associated with inflammation and not in CFF, which is a neurophysiological test that may be more related to ammonia[26]. These findings imply that in patients with impaired cognition, despite already being on lactulose and rifaximin, mechanisms other than hyperammonemia may be important to address[27].

Prior small studies by our group and others have also shown cognitive improvement in patients with HE with fecal microbiota transplant (FMT) which most likely acts on the gut milieu[19, 28, 29]. In the Phase 1 FMT capsule trial there was also improvement in cognition with additional selective changes in the psychosocial HRQOL[28]. Unlike the FMT trial, where there was a reduction in serum LBP post-FMT, we did not find a change in LBP between groups with albumin[30]. It is likely that the beneficial impact of albumin could be due to mechanisms other than gram-negative bacterial modulation and may be due to improvement in endothelial dysfunction. Altered endothelial dysfunction has been associated in prior studies with cirrhosis and overt HE[8]. Gut-based approaches such as FMT and systemic therapies such as IV albumin may be administered concomitantly to reduce endotoxemia, inflammation, and endothelial dysfunction in patients with prior HE.

It is interesting that some aspects of the benefit of albumin lasted at least a week after the infusions were stopped, which was the only post-drug timepoint studied. This included a continued increase in total serum albumin, which was associated with cognitive and HRQOL improvement and continued reduction in markers endothelial dysfunction and in IMA levels. Moreover, the reverse trend was seen in placebo-assigned patients, where IMA and ICAM-1 remained high while IL-10 remained lower. These encouraging results could be due to the relatively long half-life of albumin and may inform future trial design that could potentially permit longer intervals between albumin infusions [31].

We blinded the outcomes assessors and patients with respect to the assignment and had a priori designed steps to prevent potential fluid overload by replacing albumin with placebo if its pre-dosing value was close to 4.0 gm/dl[32, 33]. This ensured that the outcomes, especially those that are patient-reported, were as free of bias as possible. The use of normal saline as placebo could theoretically contribute towards fluid overload but the small volumes as well as the salt content in albumin would mitigate this risk. Regardless, we did not find any difference in ascites worsening or diuretic changes between or within groups.

The use of IV albumin specifically for overt and not MHE has been studied in two trials, one which was likely underpowered to show benefit, while the other open-label trial was associated with improvement in overt HE reversal[12, 13]. Two other trials in either uncomplicated ascites or in very advanced patients with cirrhosis have been published regarding outpatients who received albumin infusion[34, 35]. These two outpatient albumin trials were not specifically designed to treat cognitive impairment and did not ascertain patient-reported outcomes. Our current trial results are narrowly focused on stable outpatients with prior HE with persistent cognitive impairment despite standard of care. Since these patients often have symptoms and HRQOL impairment that is disproportionately higher than their MELD score, they are not adequately prioritized for liver transplant[36]. Therefore, the improvement of these patient-reported outcomes in this population is novel and could alleviate this burden in this underserved population.

We conclude in this double-blind, placebo-controlled, randomized clinical trial that a 5-week course of albumin therapy improves cognitive performance and psychosocial health-related quality of life compared to placebo that persists up to a week after albumin discontinuation. This improvement is accompanied by changes related to improvement in endothelial dysfunction, systemic inflammatory milieu, and albumin functionality.

### REFERENCES

[1] Bajaj J S, Wade J B, Sanyal A J. Spectrum of neurocognitive impairment in cirrhosis: Implications for the assessment of hepatic encephalopathy. Hepatology 2009; 50:2014-2021.
[2] Vilstrup H, Amodio P, Bajaj J, Cordoba J, Ferenci P, Mullen K D, et al. Hepatic encephalopathy in chronic liver disease: 2014 Practice Guideline by the American Association for the Study of Liver Diseases and the European Association for the Study of the Liver. Hepatology 2014; 60:715-735.
[3] Bajaj J S, Schubert C M, Heuman D M, Wade J B, Gibson D P, Topaz A, et al. Persistence of cognitive impairment after resolution of overt hepatic encephalopathy. Gastroenterology 2010; 138:2332-2340.
[4] Riggio O, Ridola L, Pasquale C, Nardelli S, Pentassuglio I, Moscucci F, et al. Evidence of persistent cognitive impairment after resolution of overt hepatic encephalopathy. Clin Gastroenterol Hepatol 2010; 9:181-183.
[5] Bajaj J S, Riggio O, Allampati S, Prakash R, Gioia S, Onori E, et al. Cognitive Dysfunction Is Associated With Poor Socioeconomic Status in Patients With Cirrhosis: An International Multicenter Study. Clin Gastroenterol Hepatol 2013.
[6] Bajaj J S, Wade J B, Gibson D P, Heuman D M, Thacker L R, Sterling R K, et al. The multi-dimensional burden of cirrhosis and hepatic encephalopathy on patients and caregivers. Am J Gastroenterol 2011; 106:1646-1653.
[7] Rakoski M O, McCammon R J, Piette J D, Iwashyna T J, Marrero J A, Lok A S, et al. Burden of cirrhosis on older Americans and their families: analysis of the health and retirement study. Hepatology 2012; 55:184-191.
[8] Bajaj J S, Ahluwalia V, Wade J B, Sanyal A J, White M B, Noble N A, et al. Asymmetric dimethylarginine is strongly associated with cognitive dysfunction and brain MR spectroscopic abnormalities in cirrhosis. J Hepatol 2012; 58:38-44.
[9] Shawcross D L, Davies N A, Williams R, Jalan R. Systemic inflammatory response exacerbates the neuropsychological effects of induced hyperammonemia in cirrhosis. J Hepatol 2004; 40:247-254.
[10] Shawcross D L, Wright G, Olde Damink S W, Jalan R. Role of ammonia and inflammation in minimal hepatic encephalopathy. Metab Brain Dis 2007; 22:125-138.
[11] Garcia-Martinez R, Caraceni P, Bernardi M, Gines P, Arroyo V, Jalan R. Albumin: Pathophysiologic basis of its role in the treatment of cirrhosis and its complications. Hepatology 2013.
[12] Simon-Talero M, Garcia-Martinez R, Torrens M, Augustin S, Gomez S, Pereira G, et al. Effects of intravenous albumin in patients with cirrhosis and episodic hepatic encephalopathy: a randomized double-blind study. J Hepatol 2013; 59:1184-1192.
[13] Sharma B C, Singh J, Srivastava S, Sangam A, Mantri A K, Trehanpati N, et al. Randomized controlled trial comparing lactulose plus albumin versus lactulose alone for treatment of hepatic encephalopathy. J Gastroenterol Hepatol 2017; 32:1234-1239.
[14] Ampuero J, Simon M, Montoliu C, Jover R, Serra M A, Cordoba J, et al. Minimal hepatic encephalopathy and critical flicker frequency are associated with survival of patients with cirrhosis. Gastroenterology 2015:149:1483-1489.
[15] Allampati S, Duarte-Rojo A, Thacker L R, Patidar K R, White M B, Klair J S, et al. Diagnosis of Minimal Hepatic Encephalopathy Using Stroop EncephalApp: A Multicenter U S-Based, Norm-Based Study. Am J Gastroenterol 2016; 111:78-86.
[16] Weissenborn K, Ennen J C, Schomerus H, Ruckert N, Hecker H. Neuropsychological characterization of hepatic encephalopathy. J Hepatol 2001; 34:768-773.
[17] Prasad S, Dhiman R K, Duseja A, Chawla Y K, Sharma A, Agarwal R. Lactulose improves cognitive functions and health-related quality of life in patients with cirrhosis who have minimal hepatic encephalopathy. Hepatology 2007; 45:549-559.
[18] Bajaj J S, Heuman D M, Wade J B, Gibson D P, Saeian K, Wegelin J A, et al. Rifaximin improves driving simulator performance in a randomized trial of patients with minimal hepatic encephalopathy. Gastroenterology 2011; 140:478-487 e471.
[19] Bajaj J S, Kassam Z, Fagan A, Gavis E A, Liu E, Cox I J, et al. Fecal Microbiota Transplant from a Rational Stool Donor Improves Hepatic Encephalopathy: A Randomized Clinical Trial. Hepatology 2017.
[20] Ufere N N, Satapathy N, Philpotts L, Lai J C, Serper M. Financial burden in adults with chronic liver disease: A scoping review. Liver Transpl 2022.
[21] Bajaj J S, Riggio O, Allampati S, Prakash R, Gioia S, Onori E, et al. Cognitive dysfunction is associated with poor socioeconomic status in patients with cirrhosis: an international multicenter study. Clin Gastroenterol Hepatol 2013; 11:1511-1516.
[22] Aldecoa C, Llau J V, Nuvials X, Artigas A. Role of albumin in the preservation of endothelial glycocalyx integrity and the microcirculation: a review. Ann Intensive Care 2020; 10:85.
[23] Riggio O, Ridola L, Pasquale C, Nardelli S, Pentassuglio I, Moscucci F, et al. Evidence of persistent cognitive impairment after resolution of overt hepatic encephalopathy. Clin Gastroenterol Hepatol 2011; 9:181-183.
[24] Nardelli S, Allampati S, Riggio O, Mullen K D, Prakash R, Gioia S, et al. Hepatic Encephalopathy Is Associated with Persistent Learning Impairments Despite Adequate Medical Treatment: A Multicenter, International Study. Dig Dis Sci 2016.
[25] Tapper E B, Baki J, Parikh N D, Lok A S. Frailty, Psychoactive Medications, and Cognitive Dysfunction Are Associated With Poor Patient-Reported Outcomes in Cirrhosis. Hepatology 2019; 69:1676-1685.
[26] Montagnese S, Biancardi A, Schiff S, Carraro P, Carla V, Mannaioni G, et al. Different biochemical correlates for different neuropsychiatric abnormalities in patients with cirrhosis. Hepatology 2011; 53:558-566.
[27] Tranah T H, Edwards L A, Schnabl B, Shawcross D L. Targeting the gut-liver-immune axis to treat cirrhosis. Gut 2020.
[28] Bajaj J S, Salzman N H, Acharya C, Sterling R K, White M B, Gavis E A, et al. Fecal Microbial Transplant Capsules are Safe in Hepatic Encephalopathy: A Phase 1, Randomized, Placebo-Controlled Trial. Hepatology 2019.
[29] Bloom P P, Donlan J, Torres Soto M, Daidone M, Hohmann E, Chung R T. Fecal microbiota transplant improves cognition in hepatic encephalopathy and its effect varies by donor and recipient. Hepatol Commun 2022.
[30] Bajaj J S, Salzman N, Acharya C, Takei H, Kakiyama G, Fagan A, et al. Microbial Functional Change is Linked with Clinical Outcomes after Capsular Fecal Transplant in Cirrhosis. JCI Insight 2019.
[31] Garcia-Martinez R, Caraceni P, Bernardi M, Gines P, Arroyo V, Jalan R. Albumin: pathophysiologic basis of its role in the treatment of cirrhosis and its complications. Hepatology 2013; 58:1836-1846.
[32] China L, Freemantle N, Forrest E, Kallis Y, Ryder S D, Wright G, et al. A Randomized Trial of Albumin Infusions in Hospitalized Patients with Cirrhosis. N Engl J Med 2021; 384:808-817.
[33] Wong F, Pappas S C, Curry M P, Reddy K R, Rubin R A, Porayko M K, et al. Terlipressin plus Albumin for the Treatment of Type 1 Hepatorenal Syndrome. N Engl J Med 2021; 384:818-828.
[34] Caraceni P, Riggio O, Angeli P, Alessandria C, Neri S, Foschi F G, et al. Long-term albumin administration in decompensated cirrhosis (ANSWER): an open-label randomised trial. Lancet 2018; 391:2417-2429.
[35] Sola E, Sole C, Simon-Talero M, Martin-Llahi M, Castellote J, Garcia-Martinez R, et al. Midodrine and albumin for prevention of complications in patients with cirrhosis awaiting liver transplantation. A randomized placebo-controlled trial. J Hepatol 2018; 69:1250-1259.
[36] Acharya C, Bajaj J S. Hepatic Encephalopathy and Liver Transplant: The Past, Present, and Future Towards Equitable Access. Liver Transpl 2021.

What is claimed is:

1. A method of treating cognitive impairment in a subject suffering from cirrhosis, comprising administering to the subject a therapeutically effective amount of albumin, wherein the subject does not suffer from overt hepatic encephalopathy.

2. The method of claim 1, wherein the subject has minimal hepatic encephalopathy.

3. The method of claim 1, wherein the subject has previously suffered from overt hepatic encephalopathy.

4. The method of claim 3, wherein the subject has not suffered from overt hepatic encephalopathy within one month of the administering step.

5. The method of claim 1, wherein the subject has not previously suffered from overt hepatic encephalopathy.

6. The method of claim 1, wherein the cognitive impairment is resistant to treatment with lactulose or rifaximin.

7. The method of claim 1, wherein the albumin is administered intravenously.

8. The method of claim 1, wherein the albumin is administered weekly for 3-7 weeks.

9. The method of claim 1, wherein the therapeutically effective amount is 0.5-2 g/kg of body weight.

10. The method of claim 1, wherein the subject does not have cognitive impairment caused by aging or an aging-associated disorder.

11. The method of claim 1, wherein the subject does not have dementia.
